(19) United States
(12) Patent Application Publication
(10) Pub. No.: US 2023/0017130 A1
(43) Pub. Date: Jan. 19, 2023

(54) SWALLOWABLE DEVICES FOR DRUG DELIVERY IN AN INTESTINAL TRACT

(71) Applicant: Rani Therapeutics, LLC, San Jose, CA (US)

(72) Inventor: Mir A. IMRAN, Los Altos Hills, CA (US)

(73) Assignee: Rani Therapeutics, LLC, San Jose, CA (US)

(21) Appl. No.: 17/863,806

(22) Filed: Jul. 13, 2022

Related U.S. Application Data
(63) Continuation of application No. PCT/US2021/013256, filed on Jan. 13, 2021.
(60) Provisional application No. 62/960,977, filed on Jan. 14, 2020.

(51) Int. Cl. A61M 31/00; A61K 9/00

(52) U.S. Cl. CPC A61M 31/002; A61K 9/0097; A61M 2210/1042

(57) ABSTRACT

A delivery device may include a capsule housing, at least one tissue penetrating member in a sealed compartment in the capsule housing, wherein the at least one tissue penetrating member is configured to release a payload, and an actuator in the capsule housing and at least partially outside the sealed compartment, wherein the actuator is configured to advance the at least one tissue penetrating member out of the sealed compartment.

### CROSS-REFERENCE TO RELATED APPLICATIONS

This application claims priority to U.S. Patent Application Ser. No. 62/960,977 filed Jan. 14, 2020, which is incorporated herein in its entirety by this reference.

### TECHNICAL FIELD

This invention relates generally to the field of drug delivery devices.

### BACKGROUND

Medical treatment of many conditions involves the use of drugs, which are conventionally administered to patients in various manners such as orally or through injection (e.g., subcutaneous injection). However, these modes of drug delivery have drawbacks. For example, with conventional oral administration, patients may experience gastric irritation or other discomfort, and/or drugs may undergo undesirable degradation as a result of digestion in the gastrointestinal tract. Furthermore, some therapeutic agents (e.g., large molecules) are not able to be delivered orally. As another example, injections are painful and inconvenient, and often affect patient compliance and quality of life. These problems are compounded for chronic medical conditions requiring sustained or repeated administration of drugs. Thus, there is a need for new and improved methods and devices for delivering drugs to a patient.

### SUMMARY

In some variations, a delivery device includes a capsule housing, at least one tissue penetrating member in a sealed compartment in the capsule housing, and an actuator in the capsule housing at least partially outside the sealed compartment. The at least one tissue penetrating member may be configured to release a payload or other therapeutic agent, such as a drug. The actuator may be configured to advance the at least one tissue penetrating member out of the sealed compartment. For example, in some variations, the sealed compartment may include one or more seals, where the actuator may be configured to breach at least one seal and/or advance the at least one tissue penetrating member through at least one seal.

The sealed compartment may be configured to protect the tissue penetrating member from degradation (e.g., from the environment of the gastrointestinal tract) until it is advanced into tissue, thereby substantially reducing or eliminating the risk of premature release of the drug where it is not readily absorbed. Thus, the sealed compartment may help retain the treatment efficacy of the dose of drug provided in the drug delivery device.

### BRIEF DESCRIPTION OF THE DRAWINGS

FIG. 1A depicts a schematic illustration of an example of a variation of a swallowable drug delivery device.

FIG. 1B depicts a schematic illustration of a tissue penetrating member in a sealed compartment.

FIG. 1C depicts a schematic illustration of the tissue penetrating member depicted in FIG. 1B being advanced out of the sealed compartment.

FIG. 2A depicts a schematic illustration of an example of a variation of a swallowable drug delivery device.

FIG. 2B depicts a schematic illustration of a plurality of tissue penetrating members in sealed compartments.

FIG. 3 depicts a schematic illustration of an example of a variation of a tissue penetrating member in a sealed compartment.

FIGS. 4A-4C depict schematic illustrations of examples of variations of a tissue penetrating member configured to release a drug.

FIGS. 5A and 5B depict schematic illustrations of a distal end of examples of variations of a tissue penetrating member.

FIG. 6A depicts a schematic illustration of a drug delivery device including an example of a variation of an actuator.

FIG. 6B depicts a schematic illustration of the actuator depicted in FIG. 6A after the removal of a capsule housing.

FIG. 6C depicts a schematic illustration of the actuator depicted in FIG. 6B in an expanded configuration for advancing one or more tissue penetrating members.

FIG. 7A depicts a schematic illustration of a drug delivery device including an example of a variation of an actuator.

FIG. 7B depicts a schematic illustration of the actuator depicted in FIG. 7A after the removal of a capsule housing.

FIG. 7C depicts a schematic illustration of the actuator depicted in FIG. 7B in an expanded configuration for advancing one or more tissue penetrating members.

FIG. 8A depicts a schematic illustration of a drug delivery device including an example of a variation of an actuator.

FIG. 8B depicts a schematic illustration of the actuator depicted in FIG. 8A in an expanded configuration for advancing one or more tissue penetrating members.

FIG. 9A depicts a schematic illustration of a drug delivery device including an example of a variation of an actuator.

FIG. 9B depicts a schematic illustration of the actuator depicted in FIG. 9A after the removal of a capsule housing.

FIG. 9C depicts a schematic illustration of the actuator depicted in FIG. 9B in an expanded configuration for advancing one or more tissue penetrating members.

### DETAILED DESCRIPTION

Non-limiting examples of various aspects and variations of the invention are described herein and illustrated in the accompanying drawings.

When used in the present disclosure, the terms “e.g.”, “such as”, “for example”, “examples of”, and “by way of example” indicates that a list of one or more non-limiting example(s) precedes or follows; it is to be understood that other examples not listed are also within the scope of the present disclosure.

The terms “substantially” and “about” are used herein to describe and account for small variations. For example, when used in conjunction with a numerical value, the terms can refer to a variation in the value of less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

As used herein, a range of numbers includes any number within the range, or any sub-range if the minimum and maximum numbers in the sub-range fall within the range. Thus, for example, “<9” can refer to any number less than nine, or any sub-range of numbers where the minimum of the sub-range is greater than or equal to zero and the maximum of the sub-range is less than nine.

A delivery device as described herein delivers a payload to a site, such as a site within a body (e.g., a human or other animal body). A payload can be or include one or more formulations, an electronic device, or a combination of the foregoing. A formulation may be in a powder form or in a condensed or a consolidated form, such as a tablet or microtablet. A delivery device can include one or more formulations. A formulation can include one or more agents. A wide range of agents can be used. For example, agents can be, or can include, any pharmacologically active agent (e.g., antibiotic, NSAID, angiogenesis inhibitor, neuroprotective agent, chemotherapeutic agent), a DNA or SiRNA transcript (e.g., for modifying genetic abnormalities, conditions, or disorders), a cell (e.g., produced by or from living organisms or contain components of living organisms), a cytotoxic agent, a diagnostic agent (e.g., sensing agent, contrast agent, radionuclide, fluorescent moiety, luminescent moiety, magnetic moiety), a prophylactic agent (e.g., vaccine), a nutraceutical agent (e.g., vitamin, mineral, herbal supplement), a delivery enhancing agent, a delay agent, an excipient, another substance, or any combination of two or more of the foregoing. An agent can be suitable for introduction to biological tissues. For convenience of nomenclature, delivery devices may be labeled herein as “drug delivery” devices, although more generally a delivery device may deliver a payload which may include one or more formulations as discussed above and/or electronic devices. Further for convenience of nomenclature, a payload may be referred to as a “drug” herein, although a payload may be or include one or more formulations, an electronic device, or a combination of the foregoing.

As described in further detail herein, drug delivery devices and methods may utilize a swallowable device for delivering payloads into various locations of the body. In some variations, a drug delivery device may be a swallowable device configured to deliver one or more therapeutic agents into a gastrointestinal tract. As described in further detail below, the drug delivery device may include a capsule housing, at least one tissue penetrating member configured to release a payload, and an actuator in the capsule housing and configured to advance the tissue penetrating member into tissue where the payload may be released. For example, the tissue penetrating member may include a biodegradable material that releases a drug when the tissue penetrating member degrades.

In some variations, the tissue penetrating member may be in a sealed compartment within the capsule housing, and the actuator may be at least partially outside the sealed compartment and configured to advance the tissue penetrating member out of the sealed compartment and into nearby tissue. The sealed compartment may be configured to protect the tissue penetrating member from degradation (e.g., from the environment of the gastrointestinal tract) until it is advanced into tissue. For example, the sealed compartment may be configured to protect the tissue penetrating member and thereby substantially reduce or eliminate a risk of premature release of a drug where it is not readily absorbed; thus, such a sealed compartment for the tissue penetrating member may help retain the treatment efficacy of the dose of drug provided in the drug delivery device.

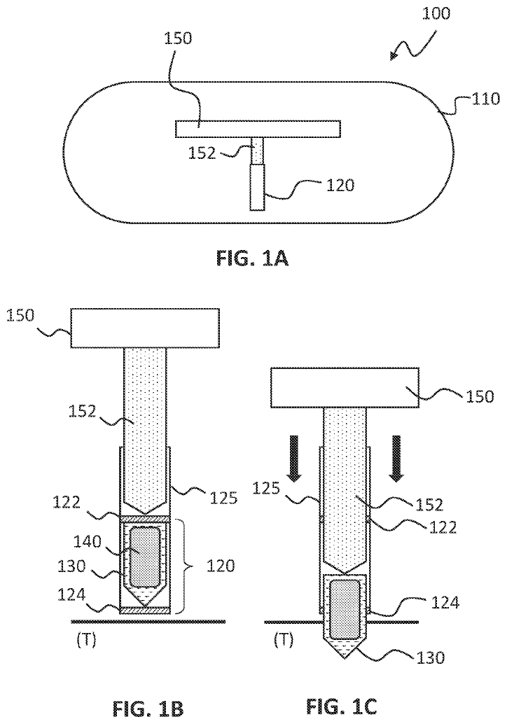

For example, as shown in the schematics of FIGS. 1A-1C, a drug delivery device 100 may include a capsule housing 110, at least one tissue penetrating member 130 in a sealed compartment 120 and configured to release a drug 140 (or other payload 140), and an actuator 150 in the capsule housing 110 that is at least partially outside the sealed compartment 120. The actuator 150 may be configured to advance the tissue penetrating member 130 out of the sealed compartment 120 and into tissue (T).

Once swallowed, the drug delivery device 100 may travel through the gastrointestinal tract, and the capsule housing 110 may degrade as the result of the environment (e.g., pH) of the gastrointestinal tract. In some variations, the actuator 150 may be prevented (e.g., with one or more biodegradable restraint features as further described below) from advancing the tissue penetrating member 130 until after the capsule housing degrades. For example, while or after the capsule housing degrades, the actuator may become exposed to environmental conditions and thereby activated to advance the tissue penetrating member 130 into the intestinal wall. However, the sealed compartment 120 may protect the tissue penetrating member 130 exposure to the same environmental conditions until the actuator is fully activated to advance the tissue penetrating member 130 into tissue, for example where the drug 140 is released. Thus, the drug delivery device with the sealed compartment 120 reduces or prevents the premature degradation of the tissue penetrating member 130 and release of the drug 140, and, for example, helps maintain full therapeutic effect of a drug dose contained in the tissue penetrating member 130.

### Capsule Housing

Generally, the capsule housing may be sized and shaped to be swallowed and pass into the gastrointestinal tract. For example, as shown in FIG. 1A, the capsule housing may be spherocylindrical (e.g., cylindrical with hemispherical or otherwise rounded ends). However, the capsule housing may have any suitable shape such as spherical or ellipsoid. The capsule housing may have rounded edges, which may help avoid difficulty swallowing and/or injury to the gastrointestinal tract during passage of the drug delivery device.

The capsule housing may include an interior volume for containing one or more other components of the drug delivery device, such as one or more sealed compartments, tissue penetrating members, and/or actuators. The capsule housing may have capsule walls defining the interior volume. In some variations, the interior volume may be substantially sealed (e.g., the capsule housing may entirely enclose its contents). The capsule housing may include one or more apertures (e.g., for permitting passage of one or more tissue penetrating members) which may be temporarily covered with a dissolvable coating or other seal. Furthermore, in some variations, the capsule housing may include one or more capsule walls forming one or more partitions of the interior capsule volume, thereby segmenting the interior capsule volume in any suitable manner.

The capsule housing may include any suitable biodegradable materials, such as one or more biodegradable polymers. The capsule housing may, for example, be formed out of such biodegradable materials, and/or include a biodegradable coating. Examples of biodegradable polymers that may be suitable for use with the methods and devices described here include, but are not limited to, hydroxypropyl methylcellulose (HPMC), lactide, glycolide, lactic acid, glycolic acid, para-dioxanone, trimethylene carbonate, caprolactone, and mixtures and copolymers thereof. In one or more embodiments, the capsule housing is formed of one or more layers of HPMC, Furthermore, in some variations the capsule housing may include an enteric outer coating to help protect the capsule housing from dissolution in the stomach prior to being passed into the intestine.

The capsule housing may be configured to degrade in whole or in part during passage in the gastrointestinal tract. For example, the capsule housing may degrade to expose at least a portion of its contents of the interior capsule volume. In some variations, the material of the capsule housing may be configured to degrade in an intestinal environment (e.g., in the small intestine) in which the pH is at least about 5.5. For example, the capsule housing may be formed from a material and/or include a coating that is configured to degrade in an environment having a pH of at least 5.5, at least 6.0, at least 6.5, at least 7.0, at least 7.1, at least 7.2, at least 7.3, at least 7.4, at least 7.5, at least 7.6, at least 7.7, at least 7.8, at least 7.9, at least 8.0, etc. In some variations, the dimensions and materials of the capsule housing may be selected such that the capsule housing (or a coating thereon) is configured to degrade over a predetermined period of time in the gastrointestinal tract, such as between about four hours and about ten hours, between about five hours and about nine hours, or between about six hours and about eight hours. The predetermined period of time may be selected based at least in part on desired location for payload delivery (e.g., stomach, small intestine, large intestine, etc.), an estimated travel rate of the capsule housing in the gastrointestinal tract due to peristalsis, and/or other factors.

The capsule housing may be configured to dissolve in its entirety, and/or the capsule housing may break apart into smaller pieces (e.g., due to dissolvable joints or seams) to facilitate easier passage through the gastrointestinal tract of the patient. In variations in which the capsule housing breaks into smaller pieces, the smaller pieces may be joined by seams in any suitable pattern (e.g., grids, rings, etc.). Such seams may include a biodegradable material, and/or may be formed by pre-stressing or otherwise weakening portions of the capsule housing. Furthermore, in variations in which the capsule housing includes one or more apertures (e.g., for passage of one or more tissue penetrating members), the one or more apertures may be covered with a dissolvable seal comprising a biodegradable material (e.g., a pH-controlled material, similar to those described above).

Specific size and/or shape characteristics of the capsule housing may be selected based on the application (e.g., volume of drug to be delivered, patient size or age, etc.). For example, in some variations the capsule housing length may range between about 0.25 inches to about 2 inches, between about 0.5 inches to about 1.5 inches, between about 0.75 inches to about 1.25 inches, etc. In some variations, the capsule housing diameter may range between about 0.1 inches to about 0.5 inches, for example.

### Sealed Compartment

In some variations, the drug delivery device may include one or more sealed compartments. A sealed compartment may, for example, function to temporarily protect at least one tissue penetrating member contained therein from environmental factors (e.g., higher pH of the intestine) which may prematurely degrade the tissue penetrating member before its advancement into tissue for payload delivery. In other words, the sealed compartment may delay the exposure of the tissue penetrating member to degrading conditions until the tissue penetrating member is advanced into the intestinal wall or other tissue by an actuator.

In some variations, a sealed compartment may include one or more seals coupled to a chamber, guide tube, or similar structure containing at least one tissue penetrating member. The one or more seals may form a fluid-tight seal to substantially prevent entry of fluids into the compartment. The sealed compartment may be breached at a suitable time to allow a tissue penetrating member contained therein to exit the tissue sealed compartment, penetrate tissue, and deliver a payload. Sealed compartments may include mechanical seals that may be breached with mechanical processes (e.g., piercing, puncturing, loosening, etc.) and/or chemical seals that may be breached with chemical processes (e.g., dissolving, other chemical degradation, etc.), as further described below.

FIGS. 1B and 1C illustrate an example of a variation of a sealed compartment 120 for a tissue penetrating member 130. As shown in FIG. 1B, the sealed compartment 120 may include a proximal seal 122 and/or a distal seal 124 at opposite ends of a guide tube or chamber 125. The proximal seal 122 and/or the distal seal 124 may include a mechanical seal such as a layer of foil or a film of a biocompatible material that may be pierced to provide access into and/or out of the sealed compartment 120. For example, the proximal seal 122 and/or the distal seal 124 may include a foil seal made of aluminum or other suitable material that provides sufficient rigidity and pierceability. In one or more embodiments, the proximal seal 122 and/or the distal seal 124 is aluminum foil about ten micrometers to about twenty micrometers thick, bonded with ethylene-vinyl acetate (EVA) or poly(ethylene-vinyl acetate) (PEVA) thermal adhesive. In some variations, the material of the proximal seal 122, the distal seal 124, and/or one or more of the walls of the compartment 120 may be biodegradable. In some variations, the other portions of the compartment 120 may include a biodegradable polymer, such as any of those described above with respect to the capsule material.

As described in further detail below, the actuator 150 may include a driving member 152 or other suitable feature arranged to cause breach of the proximal seal 122 and/or the distal seal 124 when the actuator 150 is activated. The driving member 152 may operate similar to a piston or plunger in the guide chamber 125 adjacent the sealed compartment 120. For example, as shown in FIG. 1C, the driving member of the actuator 150 may have a sharpened tip and be triggered to pierce the proximal seal 122. After piercing the proximal seal 122, the driving member may in turn urge the tissue penetrating member 130 to pierce the distal seal 124, thereby advancing the tissue penetrating member 130 out of the sealed compartment 120. In some variations, one or both of the driving member 152 and the chamber adjacent the sealed compartment 120 (and/or the sealed compartment 120) may include directional features (e.g., notches) to permit movement of the driving member 152 in one direction (e.g., a distal direction to advance the tissue penetrating member 130), and resist movement of the driving member 152 in the opposite direction, thereby restraining the driving member 152 to move in a predetermined direction.

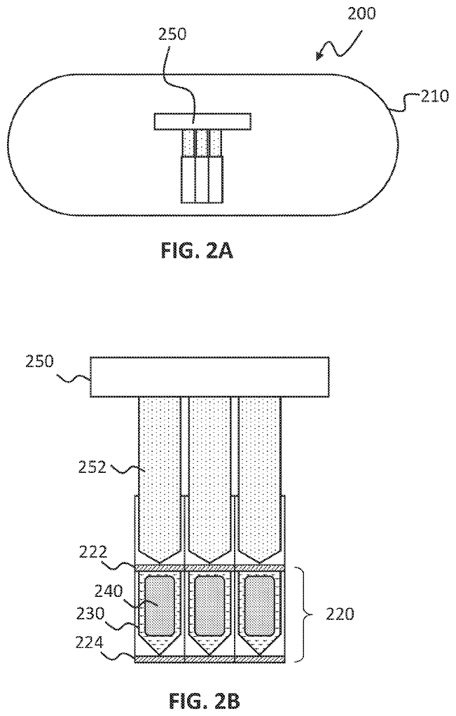

In some variations, a drug delivery device may include multiple sealed compartments for containing and protecting multiple tissue penetrating members. For example, FIGS. 2A and 2B depict a schematic of a drug delivery device 200 and an arrangement of multiple sealed compartments 220, respectively. Features of drug delivery device 200 are numbered analogously to those shown and described above with respect to the drug delivery 100 shown in FIGS. 1A-1C. Although three sealed compartments are depicted for sake of illustration, it should be understood that any suitable number (two, four, five, six or more, etc.) of sealed compartments may be included in the drug delivery device. Multiple sealed compartments may be arranged in any suitable manner, such as in a row, a ring or other perimeter, a cluster, a matrix of two or more rows and two or more columns, a corner-to-corner arrangement, or other arrangement.

Operation and function of the sealed compartments 220 may be generally similar to that shown and described above with respect to FIGS. 1A-1C. However, as shown in detail in FIG. 2B, the drug delivery device 200 may include multiple sealed compartments 220 each containing a respective tissue penetrating member 230 including a payload 240. Furthermore, an actuator 250 may include multiple driving members 252 or other suitable features, where each driving member 252 is configured to pierce a proximal seal 222 and advance a tissue penetrating member 230 out of a respective sealed compartment 220 (e.g., through a distal seal 224). Although the driving members 252 are depicted in FIG. 2B are being driven simultaneously by a common actuator 250, it should be understood that some or all of the driving members 252 may be separately and individually actuated by a respective actuator. In some variations, multiple actuators may be configured to activate at different times such that tissue penetrating members are advanced out of the sealed compartment(s) in a staged manner. Furthermore, although the variation shown in FIG. 2B includes each tissue penetrating member 230 individually contained in a respective sealed compartment 220, it should be understood that in some variations, some or all of the multiple tissue penetrating members may share a sealed compartment 220.

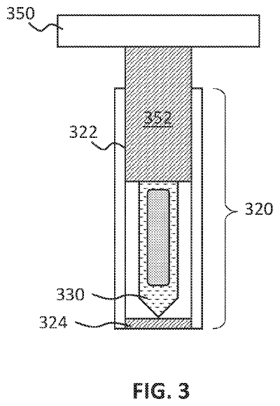

In some variations, a sealed compartment may include one or more seals formed as the result of an engineering fit between a sealing feature and at least one surface of the sealed compartment (e.g., walls of the guide tube or chamber). For example, FIG. 3 illustrates an example of a variation of an arrangement in which a sealed compartment 320 containing a tissue penetrating member 330 has a proximal seal 322 and a distal seal 324. The proximal seal 322 may be formed by an engineering fit between an outer surface of a driving member 352 of an actuator 350, and an inner surface of a wall of the sealed chamber 320. An outer diameter of the driving member 352 may, for example, be sufficiently oversized relative to the inner diameter of the compartment 320, so as to form a sufficiently fluid-tight fit. However, the relative sizes of the driving member 352 and the compartment 320 may furthermore be selected to allow the actuator force to overcome the friction in the engineering fit, so as to allow the actuator 350 to advance the tissue penetrating member 330 through the distal seal 324 and out of the sealed compartment 320.

Additionally or alternatively, a sealed compartment may include one or more seals that may dissolve or otherwise chemically degrade. For example, the sealed compartment may include a proximal seal and/or a distal seal (similar to that shown in FIG. 1B or FIG. 2B) including a biodegradable material, such as a biodegradable polymer including those described above with respect to the capsule housing. In these variations, instead of being pierced by the actuator or tissue penetrating member, a seal may dissolve as the result of environmental conditions (e.g., pH) of the intestinal tract. Furthermore, in some variations, both chemical and mechanical processes may breach one or more seals of the sealed compartment. For example, chemical degradation processes may weaken a mechanical seal over time (e.g., a predetermined delay period of time) so as to make it easier for the actuator to pierce the mechanical seal. As another example, a sealed compartment may include both at least one chemical seal and at least one mechanical seal (e.g., a chemical proximal seal and a mechanical distal seal).

In some variations, other forms of protection of the tissue penetrating member(s) from degrading conditions may additionally or alternatively be provided. For example, the sealed compartment walls, one or more seals, and/or the tissue penetrating member itself may include a protective outer coating. Such a protective outer coating may be configured to dissolve or otherwise degrade over time when in the environmental conditions of the intestinal tract. Any of the above-described types of protection (e.g., mechanical seals, chemical seals, coatings, etc.) may be combined in any suitable manner to delay the release of the payload in the tissue penetrating member(s) until the tissue penetrating member(s) are advanced into tissue for drug delivery.

### Tissue Penetrating Members

As described above, a drug delivery device may include one or multiple tissue penetrating members (e.g., microneedles) configured to release a payload such as a therapeutic agent. In some variations, the tissue penetrating member may be hollow (e.g., include a lumen or other recess) containing the payload, such as a drug. Alternatively, as described in further detail below, the tissue penetrating member may be solid (e.g., formed at least partially from a drug itself). In variations in which a drug delivery device includes multiple tissue penetrating members containing drugs, each of the tissue penetrating members may include the same or similar drug, or one or more of the tissue penetrating members may include different payloads. Furthermore, in some variations in which the payload is a therapeutic agent, a tissue penetrating member may include a preparation of multiple therapeutic agents in combination.

Generally, a tissue penetrating member may include a shaft and a tip suitable for penetrating tissue. Once placed in tissue, the tissue penetrating member may degrade due to conditions in the tissue, such that the payload is released (e.g., the tissue penetrating member degrades and is dissolved to release the payload) and, where the payload is a drug, the drug may be absorbed into the blood stream. In some variations, the tissue penetrating member may include one or more retention features such as barbs, hooks, textural features (e.g., frictional bumps or rings, etc.), to help fix the tissue penetrating member in the tissue once placed. The retention features may, for example, be arranged around the outer surface of the tissue penetrating member in a ring, helix, grid, or in any suitable pattern.

In some variations, the tissue penetrating member may include a biodegradable material so as to be dissolvable such as after penetrating tissue. Like the capsule housing described above, in some variations the tissue penetrating member may include one or more biodegradable seams to allow the tissue penetrating member to break apart into smaller pieces. The material of the tissue penetrating member may be selected to provide suitable structural properties (e.g., rigidity and/or column strength) and/or based on degradation qualities (e.g., rate). For example, the tissue penetrating member may include a biodegradable polymer such as polyethylene glycol (PEG) (e.g., injectable-grade PEG). As another example, the tissue penetrating member may additionally or alternatively include cellulose, or a sugar such as maltose.

For embodiments in which the payload includes a therapeutic agent, the tissue penetrating member may include any suitable dose of the therapeutic agent. For example, in some variations, the tissue penetrating member may include between about 0.1 mg and about 10 mg, between about 1 mg and about 8 mg, between about 1 mg and about 5 mg, or between about 1 mg and about 3 mg of a drug or other therapeutic agent. However, the specific amount of therapeutic agent may be tailored based on the type of therapeutic agent, the number of drug delivery devices intended to be taken at any particular time, the characteristics of the patient (e.g., age, weight, sex, BMI, etc.) and the like. The therapeutic agent may be formulated to achieve a desired pharmacokinetic profile. For example, in one or more embodiments in which the therapeutic agent includes basal insulin, the therapeutic agent has a formulation designed to achieve a half-life of at least twenty-four hours for the basal insulin in the formulation.

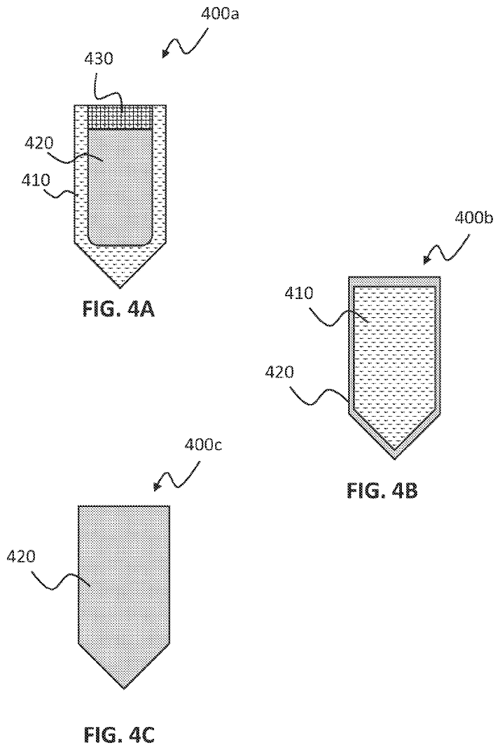

As shown in FIGS. 4A and 4B, a biodegradable material 410 may be included with a drug 420 in the tissue penetrating member in various manners. For example, as shown in FIG. 4A, a tissue penetrating member 400a may include a biodegradable material 410 forming a member body with a sharpened penetrating tip, and with a lumen or recess for receiving a drug 420. The member body may include, for example, a biodegradable polymer as described above, which may be formed into a member body through molding or other suitable techniques. The drug 420 may, for example, be a solid form (e.g., powder, tablet, cylindrical slug or other suitable shaped volume, etc.) configured to reside in the recess of the member body. For example, powder may be poured and/or packed into the recess of the tissue penetrating member. As another example, the solid form of the drug may be separately formed and then inserted into the recess of the tissue penetrating member. In other variations, the drug may be in semi-liquid, liquid, or other fluid form poured into the recess of the tissue penetrating member.

The drug 420 may further be contained in the recess of the member body with a seal 430 (e.g., heat seal, chemical seal, foil seal, etc.) that is dissolvable or otherwise degradable. Formation of the tissue penetration member may be accomplished with suitable polymer and/or pharmaceutical fabrication techniques (e.g., molding, etc.).

As another example, as shown in FIG. 4B, a tissue penetrating member 400b may include a biodegradable material 410 formed into a member body with a sharpened penetrating tip, and a coating on the member body including the drug 420. The drug 420 may, for example, be deposited as a conformal coating on the member body (e.g., with a dip, spray, or other suitable process). The drug coating may be present around the entire member body, or only a portion of the tissue penetrating member. In some variations, the drug coating may be substantially uniform in thickness, or may vary in thickness. The thickness may also be selected depending on, for example, a desired dose of drug and/or the surface area of the tissue penetrating member to be exposed for drug absorption.

In some variations, the tissue penetrating member may include a drug fabricated into the shape of the tissue penetrating member, without being combined with a biodegradable material. For example, as shown in FIG. 4C, a tissue penetrating member 400c may include a drug 420 formed into a member body with a sharpened penetrating tip, such as through shaving, molding, and/or any suitable formation techniques.

It should also be understood that a drug (or multiple drugs) may be included in a tissue penetrating member in a combination of manners. Aspects of any two or more of the above-described variations of tissue penetrating members may be combined. For example, a tissue penetrating member may include a recess containing a first drug (e.g., as shown in FIG. 4A), in addition to a drug coating including a second drug (e.g., as shown in FIG. 4B). As another example, a tissue penetrating member may be formed from a first drug (e.g., as shown in FIG. 4C) and include a recess containing a second drug (e.g., as shown in FIG. 4A). As another example, a tissue penetrating member may be formed from a first drug (e.g., as shown in FIG. 4C) and include a coating containing a second drug (e.g., as shown in FIG. 4B). As yet another example, a tissue penetrating may be formed from a first drug (e.g., as shown in FIG. 4C), include a recess containing a second drug (e.g., as shown in FIG. 4A), and include a coating containing a third drug (e.g., as shown in FIG. 4B). Alternatively to or additionally to any of the foregoing examples, a tissue penetrating member may include multiple drugs, such as two or more tablets each including a drug formulation.

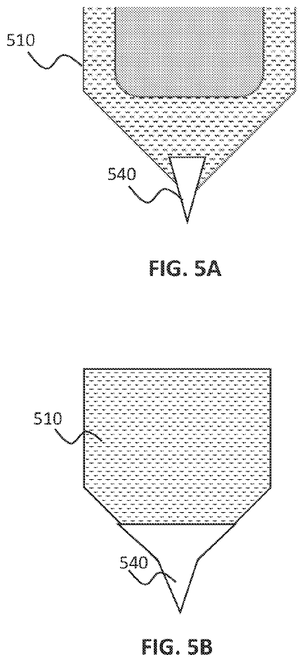

Furthermore, in some variations, a penetrating end of a tissue penetrating member may include a tip enhancement feature. The tip enhancement feature may, for example, increase a piercing capability of the tissue penetrating member by increasing the degree of pointedness of the tissue penetrating member, increasing rigidity, and/or the like. For example, as shown in FIGS. 5A and 5B, a tissue penetrating member may include a tip enhancement feature 540 that provides a more acutely angled or pointed leading end for penetrating tissue more readily. Such a tip enhancement feature 540 may be made, for example, from a relatively rigid material such as a metal (e.g., magnesium) that is better able to be formed with a sharpened shape and/or retain its sharpened shape compared to a tip formed from the biodegradable polymer alone due to differing material properties.

The tip enhancement feature 540 may be coupled to the distal end of the tissue penetrating member. For example, as shown in FIG. 5A, the tip enhancement feature 540 may be at least partially embedded in the distal end of the tissue penetrating member. In some variations, the tip enhancement feature 540 may be formed separately (e.g., molded) and inserted into the distal end of the tissue penetrating member. Alternatively, in some variations, the tip enhancement feature 540 may be formed by overmolding (comolding) a biodegradable material 510 over the tip enhancement feature 540. Generally, the retention of the tip enhancement feature 540 within the distal end of the tissue penetrating member may be improved with frictional or textural features, interlocking features, an interference fit, etc.

As another example, as shown in FIG. 5B, the tip enhancement feature 540 may additionally or alternatively be coupled to an outer surface of the distal end of the tissue penetrating member, like a spiked cap. Like the variation described above with respect to FIG. 5A, the tip enhancement feature 540 shown in FIG. 5B may be separately formed and subsequently coupled to the distal end of the tissue penetrating member.

Although the examples of variations of tissue penetrating members shown in FIGS. 4A-4C and FIGS. 5A and 5B include a shaft and a single pointed tip, it should be understood that other variations of tissue penetrating member may have other suitable shapes (e.g., multiple prongs or spikes, angled non-uniformly such as in a shape of a quill-tip pen or a ramp, etc.).

### Actuator

As described above, the drug delivery device may include one or more actuators coupled to at least one tissue penetrating member. The one or more actuators may be configured to advance at least one tissue penetrating member into tissue (e.g., intestinal wall). For example, as described above with respect to FIGS. 1B and 1C, an actuator may be configured to advance a tissue penetrating member out of a sealed compartment. In some variations, the actuator may be triggered to transition from a first state in which the actuator is prevented from actuating the tissue penetrating member, to a second state in which the actuator actuates the tissue penetrating member.

For example, in some variations the first actuator state may be maintained by one or more restraints or other suitable release features. In other words, the restraint may substantially prevent the actuator from advancing the tissue penetrating member, and the absence of the restraint may cause the actuator to transition to the second state, or activate the actuator to advance the tissue penetrating member. The restraint may, for example, include a biodegradable material that degrades in the intestinal environment such that after a predetermined period of time the restraint is removed, thereby allowing the actuator to advance the tissue penetrating member. For example, the restraint may be configured to degrade in an intestinal environment (e.g., in the small intestine) in which the pH is at least about 5.5. For example, at least a portion of the restraint may be configured to degrade in an environment having a pH of at least 5.5, at least 6.0, at least 6.5, at least 7.0, at least 7.1, at least 7.2, at least 7.3, at least 7.4, at least 7.5, at least 7.6, at least 7.7, at least 7.8, at least 7.9, at least 8.0, etc.

In some variations, the actuator may include an expandable device, where expansion of the expandable device is configured to actuate the tissue penetrating member. For example, the actuator may include a driving member, and an expandable device configured to actuate the driving member that advances a tissue penetrating member. The expandable device and/or portions of the actuator may include biodegradable materials. For example, the expandable device, driving members, and/or other portions of the actuator may include a biodegradable material with sufficient rigidity such as cellulose and poly(vinyl alcohol) (PVA).

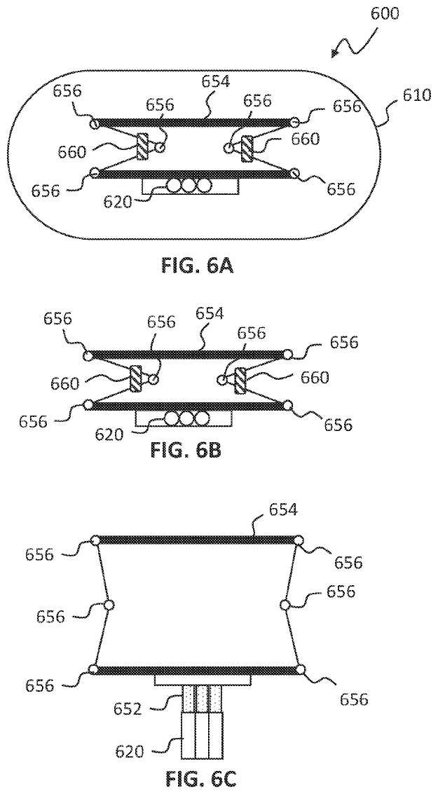

For example, FIGS. 6A-6C illustrate operation of an actuator including at least one driving member and at least one expandable device configured to actuate the driving member. As shown in FIG. 6A, a drug delivery device 600 may include a capsule housing 610, with an actuator including an expandable device 654. The expandable device 654 includes expanding struts similar to a scissor jack or lift jack mechanism. For example, the expanding struts may be joined at pivot points 656, which may include passive hinges and/or springs (e.g., torsion springs) to help actuate expansion of the expandable device 654.

As shown in FIG. 6A, the expandable device 654 is constrained in a loaded, collapsed configuration by one or more restraints 660 (two restraints 660 are illustrated in FIG. 6A by way of example). The restraints 660 may, for example, include bands, straps, or the like coupled to the expanding struts to hold the expandable device in the collapsed configuration. Although the restraints 660 are shown as coupled to internal struts, it should be understood that the restraints may couple to or around any suitable portion of the expandable device (e.g., around the entire device) so as to constrain the expandable device 654 in a collapsed state. Furthermore, the drug delivery device may include other variations of restraints, including for example braces, clips, bags surrounding the expandable device, etc.

The restraints 660 may include a biodegradable material, such that degradation of the restraints (e.g., in the gastrointestinal environment) may cause the eventual release of the expandable device. For example, as shown in FIG. 6B, degradation of the capsule housing 610 may expose the restraints 660 to gradually dissolve (while tissue penetrating members remain protected within a sealed compartment 620).

After the restraints 660 are sufficiently removed in the degradation process, the expandable device 654 may transition to its expanded state shown in FIG. 6C. Alternatively, in some variations the restrained expandable device 654 may be expelled from the capsule housing 610, such with a spring release triggered by environmental conditions. One or more tissue penetrating devices in one or more sealed compartments 620 are coupled via driving members 652 to the expandable device 654 (e.g., with an additional torsional spring (not shown)) such that the expansion of the expandable device 654 causes the driving members 652 to advance the tissue penetrating devices out of the sealed compartment(s) 620 (e.g., as described above). The tissue penetrating members may be advanced into tissue, where they release a payload (e.g., drug into a patient for therapeutic effect). Following the delivery of the tissue penetrating devices into tissue, at least some of the other components of the actuator of FIGS. 6a-6c may continue to dissolve and/or be passed through the gastrointestinal tract of the patient.

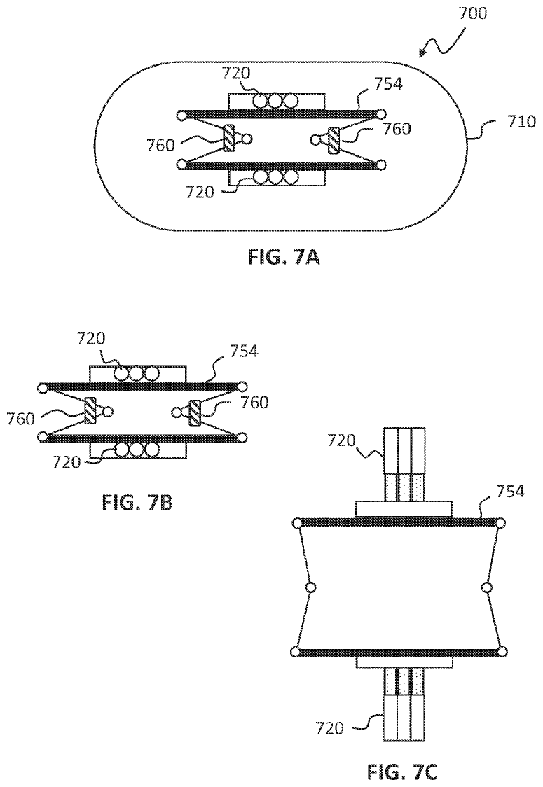

In some variations, multiple separate arrangements of tissue penetrating members may be coupled to the actuator (or to multiple respective actuators). For example, an expandable device 754 in a drug delivery device 700 depicted in FIG. 7A is similar to the expandable device 654 described above with respect to FIGS. 6A-6C, except that two chambers or sealed compartments 720 are coupled to the expandable device 754. As shown in FIG. 7A, for example, the two sealed compartments 720 are coupled on opposite sides of the expandable device 754. After the capsule housing dissolves, the restraints 760 may be exposed and subject to degradation (while the sealed compartments 720 protect the tissue penetrating members contained therein) as shown in FIG. 7B. Alternatively, in some variations the restrained expandable device 754 may be expelled from the capsule housing 710, such with a spring release triggered by environmental conditions. Upon degradation or other removal of the restraints 760, the expandable device 754 may expand, thereby urging the sealed compartments 720 in opposite directions, such as due to torsional spring action of one or more torsional springs (e.g., one, two, three, or more torsional springs). Although two opposing sealed compartments 720 are shown in FIG. 7A, it should be understood that an expandable device may include three, four, or any suitable number of multiple sealed compartments that expand outwardly. Driving members actuated by the expandable device 754 and/or others of the actuators may advance the tissue penetrating members from the sealed compartments 720 into tissue, as described above. Following the delivery of the tissue penetrating devices into tissue, at least some of the other components of the actuator may continue to dissolve and/or be passed through the gastrointestinal tract of the patient.

Although a drug delivery device is shown with two sealed compartments 720 in FIGS. 7A, 7B, and one sealed compartment 620 in FIGS. 6A-6C, additional sealed compartments 620 or 720 may be used.

Furthermore, other variations of expandable devices may additionally or alternatively be included in the drug delivery device, and at least one sealed compartment containing one or more tissue penetrating members may be arranged on the expandable device(s) in any suitable manner.

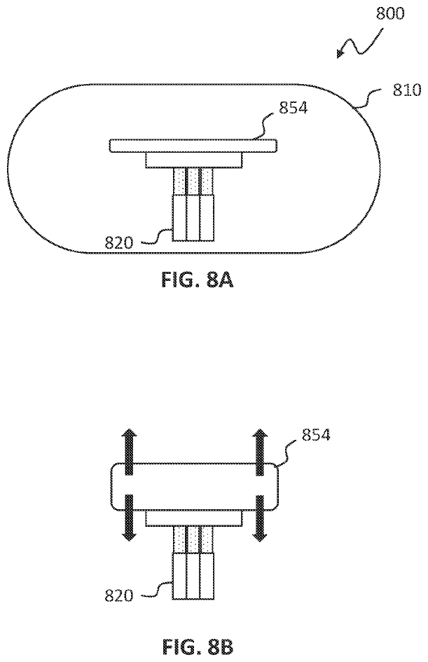

For example, as shown in FIGS. 8A and 8B, in some variations such as the drug delivery device 800, the expandable device may include at least one inflatable device 854 such as a balloon. The inflatable device 854 may include, for example, a suitable polymer such as PET, polyethylene, or polyimide. The inflatable device 854 may be arranged in a capsule housing 810, and may configured to transition from a collapsed (or partially collapsed) state shown in FIG. 8A to an expanded state shown in FIG. 8B. The transition may occur, for example, in response to a degradation of the capsule housing 810. Alternatively, in some variations the collapsed inflatable device 854 may be expelled from the capsule housing 810, such with a spring release triggered by environmental conditions. One or more arrangements of sealed compartments 820 including at least one tissue penetrating member may be coupled to the inflatable device 854 such that expansion of the inflatable device 854 actuates the tissue penetrating member(s) (e.g., via one or more driving members in a chamber).

In some variations, expansion of the inflatable device 854 may be accomplished as the result of a rapid influx of a suitable gas from a chemical reaction. For example, the inflatable device 854 may include multiple compartments separating reactants that, when mixed, produce a pneumatic output sufficient for inflating the inflatable device 854. The inflatable device 854 may further include a controllable valve or seal that, when opened, allows the reactants to mix and produce a gas for expanding the inflatable device 854. The valve or seal may function as a restraint for the actuator such that opening or absence of the element separating the compartments in turn activates the inflatable device 854. Alternatively, in some variations, the reactants may be located in another set of compartments that is separate from, but fluidically coupled to, the inflatable device 854 such that the output of the resulting reaction may flow into the inflatable device 854.

Any suitable combination of reactants may be used to produce the expanding gas. For example, one compartment may include a carbonate (e.g., metal carbonate) and another compartment may include an acid, whereby the combination of the two reactants produces a carbon dioxide gas.

Although the example of FIGS. 8A and 8B includes one arrangement of tissue penetrating members on one side of the inflatable device, it should be understood that in other variations, any suitable number of sealed compartments and/or tissue penetrating members may be arranged in any suitable manner around the inflatable device. For example, multiple tissue penetrating members may be arranged circumferentially around the inflatable device 854 and/or axially along the inflatable device 854. The tissue penetrating members may be distributed equally (e.g., generally equidistant from one another) and/or unequally.

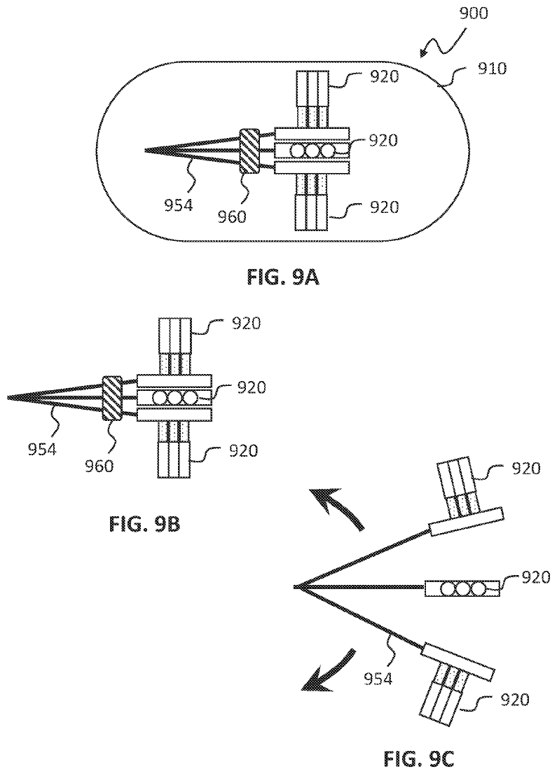

Another example of an expandable device for actuating tissue penetrating members is depicted in FIGS. 9A-9C. As shown in FIG. 9A, a drug delivery device 900 may include a capsule housing 910 with an expandable device including expandable arms 954. Coupled to each expandable arm 954 is an arrangement of sealed compartments 920 (one or more on each expandable arm 954, with three groupings of three sealed compartments 920 on each expandable arm 954 illustrated in FIGS. 9A-9C by way of example) including one or more tissue penetrating members. The expandable arms 954 may be biased toward an open or expanded configuration and temporarily restrained with a restraint 960. Similar to that described above, the restraint 960 may include a biodegradable material.

Accordingly, in some variations, after degradation of the capsule housing 910, the restraint 960 may be exposed as shown in FIG. 9B to conditions (e.g., intestinal conditions) that cause the restraint 960 to gradually biodegrade. Alternatively to the capsule housing 910 degrading, in some variations the restrained expandable device may be expelled from the capsule housing 910, such as with a spring release triggered by environmental conditions. The dissolving of the restraint 960 causes release of the expandable arms 954, thereby permitting the expandable arms 954 to transition to an expanded configuration.

The expandable arms 954 may be configured so as to urge the tissue penetrating members outward (e.g., radially outward) when the expandable arms are in the expanded configuration. For example, as shown in FIG. 9A, the expandable arms 954 may generally pivot radially outward. As another example, an expandable device may include an expandable ring comprises radially expanding struts or any other suitable structure. The bias toward the expanded configuration may be accomplished to inherent shape formation of the expandable arms themselves, bias in any connecting struts between the expandable arms, spring elements coupled to the expandable arms, and/or the like. In some variations, when in the expanded configuration, the expandable arms 954 may be configured to expand outward to similar radial distances), though in some variations at least some of the expandable arms 954 may be configured to expand outward to different radial distances (e.g., due to at least some of the expandable arms 954 having different lengths and/or pivoting joints of different stiffnesses, etc.). Furthermore, in some variations, the expandable arms 954 may be configured to expand outward at similar rates, or at some of the expandable arms 954 may be configured to expand outward at different rates.

Although the variation shown in FIGS. 9A-9C includes an expandable device with three expandable arms, it should be understood that in other variations, the expandable device may include any suitable number of expandable arms (e.g., two, three, four, five, six, or more) arranged to expand relative to one another. In some variations, the expandable device may include multiple expandable arms that are generally equally circumferentially distributed (e.g., three expandable arms arranged circumferentially 120 degrees apart from one another, four expandable arms arranged circumferentially 90 degrees apart from one another, etc.). Alternatively, the expandable device may include multiple expandable arms that are not equally circumferentially distributed. Furthermore, multiple arrangements of expandable arms may be included in a drug delivery device (e.g., arranged circumferentially and/or axially within the capsule housing volume).

Furthermore, any of the above-described types of actuators may be combined in any suitable manner to advance the tissue penetrating members into tissue for payload delivery.

### Therapeutic Agents

The methods and devices herein may be used to deliver various kinds of formulations (e.g., therapeutic agents). In some variations, drugs that would otherwise be injected (e.g., due to chemical breakdown in the presence of digestive juices) may be configured to be released from the tissue penetrating member for delivery via drug delivery devices such as those described herein. In some variations, the drug delivery device may be configured to deliver large molecule peptides and/or proteins. For example, the drug delivery device may be configured to deliver insulin and insulin-related compounds, glucagon-like peptides (e.g., GLP-1, exenatide, etc.), growth hormones (e.g., IGF and/or other growth factors), parathyroid hormones, interferons, chemotherapeutic agents (e.g., interferon), etc. A therapeutically effective dose to be included in the drug delivery device may be determined based on patient characteristics such as age, weight, sex, BMI, etc.

Furthermore, in some variations, orally administered drugs may be included in the drug delivery device. For example, the drug delivery device may include antibiotics (e.g., penicillin, erythromycin, etc.), antivirals (e.g., protease inhibitors), anti-seizure agents (e.g., furosemide, dilantin, etc.), NSAIDs (e.g., ibuprofen), immune suppression agents and/or anti-parasitic agents (e.g., anti-malarial agents). Other orally administered drugs such as painkillers, anti-inflammatories, anti-hypertensive drugs, etc. may additionally or alternatively be included in the drug delivery device. However, any suitable kind of drugs, including parenteral drugs administered non-orally, may be delivered by the drug delivery device.

Generally, a delivery device may include a capsule housing, at least one tissue penetrating member in a sealed compartment in the capsule housing, and an actuator in the capsule housing and at least partially outside the sealed compartment such that the actuator is configured to advance the at least one tissue penetrating member out of the sealed compartment. The tissue penetrating member may be configured to release a therapeutic agent. In some variations, the sealed compartment may provide protection for the at least one tissue penetrating member against release of a payload before the tissue penetrating member is advanced into tissue for release into the tissue.

One or more components of the delivery device may include a biodegradable material (e.g., biodegradable polymer). For example, the capsule housing may include a biodegradable polymer. As another example, the one or more tissue penetrating members may include a biodegradable polymer. In some variations, for example, a tissue penetrating member may include a biodegradable polymer surrounding a volume of a drug, or a biodegradable polymer having a coating including a drug. However, in other variations the tissue penetrating member may include a drug formed into a penetrating member without a biodegradable material.

The sealed compartment may be sealed in various manners. For example, in some variations, the sealed compartment may include a first seal, such as a seal arranged at a proximal end of the sealed compartment. Furthermore, the sealed compartment may include a second seal, such as a seal arranged at a distal end of the sealed compartment. In some variations, the actuator may be configured to advance the at least one tissue penetrating member through the first seal. For example, the actuator may include a driving member configured to maneuver within the sealed compartment and advance the tissue penetrating member after piercing the second seal. For example, the first seal and/or the second seal may include a mechanical seal such as foil made of aluminum or other suitable material.

The first seal and/or the second seal may be formed in various suitable manners. For example, one or both seals may be formed by an engineering fit (e.g., transition fit or interference fit) between a sealing feature and at least one surface of the sealed compartment. For example, in some variations the actuator may include the sealing feature, such as a driving member having an outer diameter sufficiently oversized relative to the inner walls of the sealed compartment so as to form a seal.

In some variations, the delivery device may include multiple tissue penetrating members. For example, each of the multiple tissue penetrating members may be in a respective sealed compartment in the capsule housing. In this example, the delivery device may further include multiple driving members, each configured to advance a respective tissue penetrating member. Alternatively, some or all of the tissue penetrating members may be arranged in a shared sealed compartment and/or may be advanced by a common driving member or other actuator feature.

The actuator may, in some variations, include a driving member and an expandable device configured to actuate the driving member. The delivery device may include one or more restraints, where the state of the restraint selectively activates the expandable device. For example, the restraint may include a biodegradable material, where degradation of the restraint is configured to activate the expandable device, thereby actuating the driving member (e.g., to advance the tissue penetrating member). The expandable device may include any suitable mechanism, such as a spring, an inflatable device such as a balloon, a lever, expanding arms, etc.

Generally, in some variations, a method of delivering a payload to tissue of a patient includes swallowing a delivery device comprising a capsule housing, at least one tissue penetrating member configured to release a payload and arranged in a sealed compartment in the capsule housing, and an actuator in the capsule housing and at least partially outside the sealed compartment. The actuator may be configured to advance the at least one tissue penetrating member out of the sealed compartment to release the payload. In some variations, a tissue penetrating member may include a biodegradable polymer surrounding a volume of a drug, or a biodegradable polymer having a coating including a drug. However, in other variations the tissue penetrating member may include a drug formed into a penetrating member. Other suitable variations of delivery devices, such as any of the delivery devices described herein, may also be swallowed and used in the method.

The sealed compartment in the swallowed device may be sealed in various manners. For example, in some variations, the sealed compartment may include a first seal, such as a seal arranged at a proximal end of the sealed compartment. Furthermore, the sealed compartment may include a second seal, such as a seal arranged at a distal end of the sealed compartment. In some variations, the actuator may be configured to advance the at least one tissue penetrating member through the first seal. For example, the actuator may include a driving member configured to maneuver within the sealed compartment and advance the tissue penetrating member after piercing the second seal. For example, the first seal and/or the second seal may include a mechanical seal such as foil made of aluminum or other suitable material.

The first seal and/or the second seal may be formed in various suitable manners. For example, one or both seals may be formed by an engineering fit (e.g., transition fit or interference fit) between a sealing feature and at least one surface of the sealed compartment. For example, in some variations the actuator may include the sealing feature, such as a driving member having an outer diameter sufficiently oversized relative to the inner walls of the sealed compartment so as to form a seal.

In some variations, the swallowed delivery device may include multiple tissue penetrating members. For example, each of the multiple tissue penetrating members may be in a respective sealed compartment in the capsule housing.

The actuator in the swallowed delivery device may, in some variations, include a driving member and an expandable device configured to actuate the driving member. The delivery device may include one or more restraints, where the state of the restraint selectively activates the expandable device. For example, the restraint may include a biodegradable material, where degradation of the restraint is configured to activate the expandable device, thereby actuating the driving member (e.g., to advance the tissue penetrating member). The expandable device may include any suitable mechanism, such as a spring, an inflatable device such as a balloon, a lever, expanding arms, etc.

Generally, in some variations, a method of delivering a payload to tissue of a patient includes swallowing a delivery device comprising a capsule housing, at least one tissue penetrating member configured to release a payload and arranged in a sealed compartment in the capsule housing, and an actuator in the capsule housing. The method further includes allowing the capsule housing to degrade in the presence of an intestinal environmental condition, and allowing the actuator to advance the at least one tissue penetrating member out of the sealed compartment after the capsule housing is degraded, thereby releasing the payload. In some variations, a tissue penetrating member may include a biodegradable polymer surrounding a volume of a drug, or a biodegradable polymer having a coating including a drug. However, in other variations the tissue penetrating member may include a drug formed into a penetrating member. Other suitable variations of delivery devices, such as any of the delivery devices described herein, may also be swallowed and used in the method.

In some variations, the actuator may be arranged at least partially outside of the sealed compartment. Furthermore, in some variations the actuator may include an expandable device and a restraint, the allowing the actuator to advance the at least one tissue penetrating member may include allowing the restraint to degrade and activate the expandable device. The expandable device may include any suitable mechanism, such as a spring, an inflatable device such as a balloon, a lever, expanding arms, etc.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A delivery device comprising:
a capsule housing;
at least one tissue penetrating member in a sealed compartment in the capsule housing, wherein the at least one tissue penetrating member is configured to release a payload; and
an actuator in the capsule housing and at least partially outside the sealed compartment, wherein the actuator is configured to advance the at least one tissue penetrating member out of the sealed compartment.

2. The delivery device of claim 1, wherein the sealed compartment comprises a first seal, and wherein the actuator is configured to advance the at least one tissue penetrating member through the first seal.

3. The delivery device of claim 2, wherein the sealed compartment comprises a second seal.

4. The delivery device of claim 3, wherein the actuator comprises a driving member configured to pierce the second seal.

5. The delivery device of claim 3, wherein second seal is formed by an engineering fit between a sealing feature and at least one surface of the sealed compartment.

6. The delivery device of claim 5, wherein the actuator comprises the sealing feature.

7. The delivery device of claim 3, wherein at least one of the first seal and the second seal comprises foil.

8. The delivery device of claim 3, wherein at least one of the first seal and the second seal comprises a biodegradable material.

9. The delivery device of claim 1, comprising a plurality of tissue penetrating members.

10. The delivery device of claim 9, wherein each of the plurality of tissue penetrating members is in a respective sealed compartment in the capsule housing.

11. The delivery device of claim 9, comprising a plurality of driving members, wherein each driving member is configured to advance a respective tissue penetrating member.

12. The delivery device of claim 1, wherein the actuator comprises a driving member and an expandable device configured to actuate the driving member.

13. The delivery device of claim 12, further comprising a restraint, wherein absence of the restraint activates the expandable device.

14. The delivery device of claim 13, wherein the restraint comprises a biodegradable material, and wherein degradation of the restraint is configured to activate the expandable device.

15. The delivery device of claim 12, wherein the expandable device comprises a spring.

16. The delivery device of claim 12, wherein the expandable device comprises an inflatable device.

17. The delivery device of claim 12, wherein the expandable device comprises one or more expandable arms.

18. The delivery device of claim 1, wherein the capsule housing comprises a biodegradable polymer.

19. The delivery device of claim 1, wherein the at least one tissue penetrating member comprises a biodegradable material surrounding a drug.

20. The delivery device of claim 1, wherein the at least one tissue penetrating member comprises a drug formed into a penetrating member.

21. The delivery device of claim 1, wherein the at least one tissue penetrating member comprises a biodegradable material comprising a coating comprising a drug.

22. A method of delivering a payload to tissue of a patient, the method comprising:
swallowing a delivery device comprising a capsule housing, at least one tissue penetrating member configured to release a payload and arranged in a sealed compartment in the capsule housing, and an actuator in the capsule housing and at least partially outside the
sealed compartment, wherein the actuator is configured to advance the at least one tissue penetrating member out of the sealed compartment to release the payload.

23-33. (canceled)

34. A method of delivering a payload to tissue of a patient, the method comprising:
swallowing a delivery device comprising a capsule housing, at least one tissue penetrating member configured to release a payload and arranged in a sealed compartment in the capsule housing, and an actuator in the capsule housing;
allowing the capsule housing to degrade in the presence of an intestinal environmental condition; and
allowing the actuator to advance the at least one tissue penetrating member out of the sealed compartment after the capsule housing is degraded, thereby releasing the payload.

35-40. (canceled)
